(19) United States
(12) Patent Application Publication
(10) Pub. No.: US 2026/0224306 A1
(43) Pub. Date: Aug. 6, 2026

(54) SOFT ROBOTIC ARM FOR IN SITU 3D BIOPRINTING AND SURGERY

(71) Applicant: NewSouth Innovations Pty Limited, Sydney (AU)

(72) Inventors: Thanh Nho DO, Sydney (AU); Mai Thanh THAI, Sydney (AU); Phuoc Thien PHAN, Sydney (AU); Nigel Hamilton LOVELL, Sydney (AU); James Jonathon DAVIES, Sydney (AU); Chi Cong NGUYEN, Sydney (AU)

(73) Assignee: NewSouth Innovations Pty Limited, Sydney (AU)

(21) Appl. No.: 19/150,359

(22) PCT Filed: Jan. 23, 2024
(86) PCT No.: PCT/AU2024/050038

§ 371 Date: Jul. 23, 2025

(30) Foreign Application Priority Data
Jan. 23, 2023 (AU) 2023900151

(51) Int. Cl. A61B 34/30; A61B 34/00; A61B 90/00

(52) U.S. Cl. CPC A61B 34/30; A61B 34/71; A61B 90/361; A61B 2034/301; A61B 2034/305

(57) ABSTRACT

An apparatus comprises a soft robotic arm and a three-dimensional (3D) bioprinter. The soft robotic arm has actuators for controlling movements of a distal end of the soft robotic arm. The 3D bioprinter is attached to the distal end of the soft robotic arm, and has a print nozzle and soft microtubule artificial muscles (SMAMs) for controlling movements of the print nozzle.

The present application relates to Australian Provisional Patent Application No. 2023900151, the contents of which are incorporated herein by reference in their entirety.

### TECHNICAL FIELD

The present invention relates to a soft robotic arm for in situ 3D bioprinting and minimally invasive surgery such as endoscopic operation and ear-nose-throat (ENT) surgery. The present invention also relates to a method of controlling the soft robotic arm, and to a computer program product including a computer readable medium having recorded thereon a computer program for controlling the soft robotic arm. The present invention also relates to a method of controlling a 3D bioprinter mounted on the soft robotic arm, and to a computer program product including a computer readable medium having recorded thereon a computer program for controlling the 3D bioprinter mounted on the soft robotic arm.

### BACKGROUND

Three-dimensional (3D) bioprinting technology offers great potential in the treatment of tissue and organ damage. Conventional 3D bioprinters generally uses a large form factor desktop bioprinter to create in vitro 3D constructs before introducing the 3D constructs into a patient's body. This conventional 3D bioprinting however poses several drawbacks such as surface mismatches between the organ and the 3D constructs, structural damage to the organ, and potential contamination of the 3D constructs due to transport and large open-field surgery.

There is a need to overcome or ameliorate one or more of the above drawbacks.

### SUMMARY

It is an object of the present invention to substantially overcome, or at least ameliorate, one or more disadvantages of existing arrangements.

Disclosed are arrangements which seek to address the above problems by providing an in vivo 3D bioprinter and a soft robotic arm, on which the 3D bioprinter is mounted, to provide better controls over the position of the 3D bioprinter over a surface of an organ.

According to a first aspect of the present disclosure, there is provided an apparatus comprising: a soft robotic arm having actuators for controlling movements of a distal end of the soft robotic arm; and a three-dimensional (3D) bioprinter attached to the distal end of the soft robotic arm, the 3D bioprinter having a print nozzle and soft microtubule artificial muscles (SMAMs) for controlling movements of the print nozzle.

According to a second aspect of the present disclosure, there is provided a method of controlling the soft robotic arm of the above aspect, the method comprising: receiving a user input to move the distal end of the soft robotic arm to a direction; determining lengths of the respective actuators to move to the direction; and transmitting control signals to actuate the hydraulic system based on the determined lengths of the actuators.

According to a third aspect of the present disclosure, there is provided a method of controlling the 3D bioprinter of the above aspect, the method comprising: receiving a predetermined path; determining a sequence of movements of the print nozzle based on the predetermined path; determining a sequence of lengths of respective SMAMs based on the determined movement sequence; and transmitting control signals to actuate the hydraulic system based on the determined sequence of lengths.

Other aspects are also disclosed.

### BRIEF DESCRIPTION OF THE DRAWINGS

At least one embodiment of the present invention will now be described with reference to the drawings, in which:

FIGS. 1A and 1B show a 3D bioprinter and a soft robotic arm according to an embodiment of the present disclosure;

FIG. 1C shows an alternative embodiment for controlling the 3D bioprinter and the soft robotic arm of FIGS. 1A and 1B;

FIG. 2A shows the robotic arm of FIGS. 1A and 1B;

FIG. 2B shows the movement of the robotic arm of FIG. 2A with the 3D bioprinter mounted on the robotic arm;

FIG. 2C shows an alternative embodiment of the robotic arm of FIGS. 1A and 1B;

FIGS. 3A and 3B show the 3D bioprinter mounted on the robotic arm of FIG. 2A;

FIGS. 4A and 4B show the 3D bioprinter of FIGS. 1A and 1B;

FIG. 5 is a flow diagram of a method of controlling the robotic arm of FIG. 2A;

FIGS. 6A and 6B show the control parameters of the robotic arm of FIG. 2A;

FIG. 7 is a flow diagram of a method of controlling the 3D bioprinter of FIGS. 4A and 4B;

FIGS. 8A to 8D show the control parameters of the 3D bioprinter of FIGS. 4A and 4B;

FIGS. 9A to 9C show a hysteresis modelling of an actuator of the 3D bioprinter of FIGS. 4A and 4B;

FIGS. 9D to 9E show the hysteresis compensation for a single actuator of the 3D bioprinter and FIGS. 9F to 9H show the tracking performance of the print nozzle of the 3D bioprinter;

FIGS. 10A to 10D show an alternative control method of the 3D bioprinter and example performance of the alternative control method;

FIGS. 11A and 11B form a schematic block diagram of a general purpose computer system upon which arrangements described can be practiced;

FIG. 12 shows the printing performance of a prototype of the 3D bioprinter; and

FIG. 13 shows an endoscopic surgery using the soft robotic arm and 3D bioprinter of the present disclosure.

### DETAILED DESCRIPTION

Where reference is made in any one or more of the accompanying drawings to steps and/or features, which have the same reference numerals, those steps and/or features have for the purposes of this description the same function(s) or operation(s), unless the contrary intention appears.

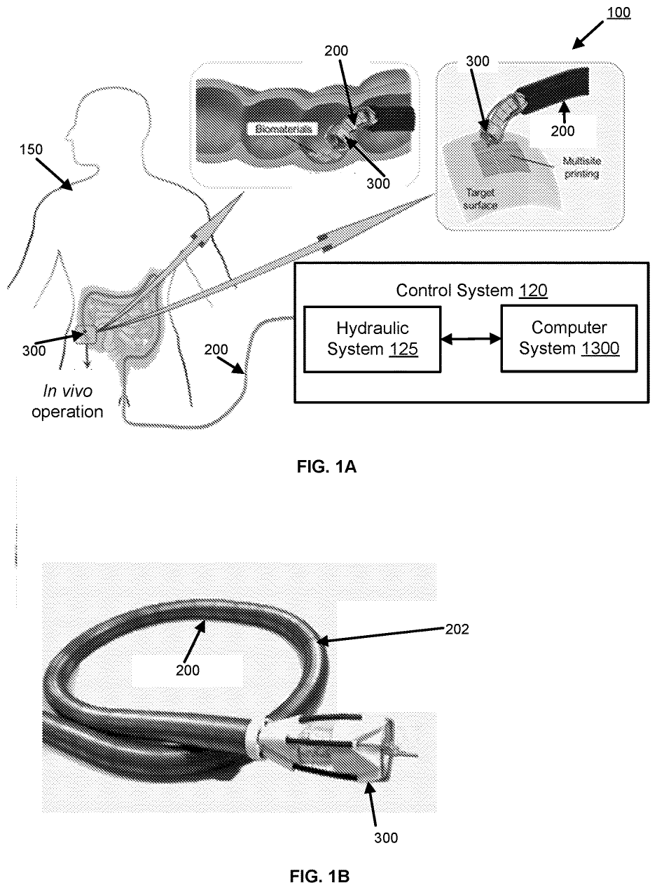

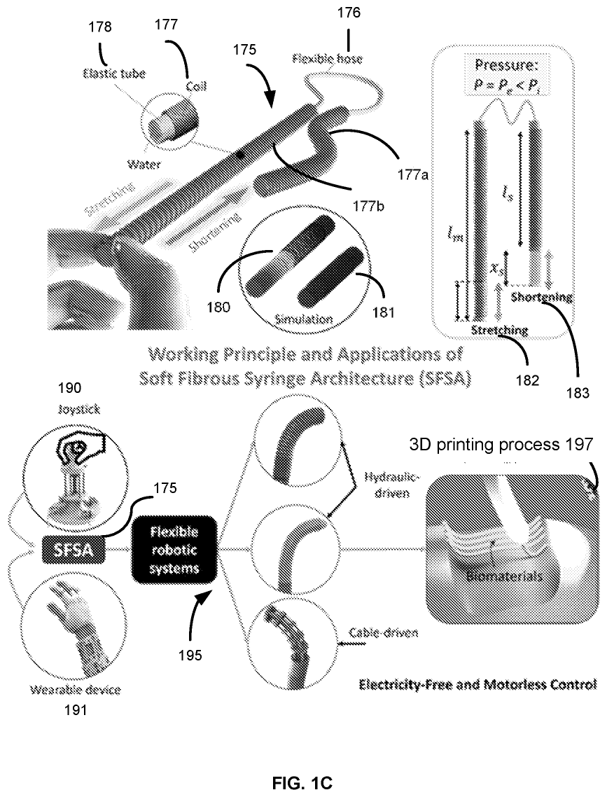

FIG. 1 shows apparatus 100 for performing in situ 3D bioprinting. Apparatus 100 includes soft robotic arm 200, 3D bioprinter 300, and control system 120. Control system 120 includes hydraulic system 125 and computer system 1300.

Hydraulic system 125 controls the movement of both soft robotic arm 200 and 3D bioprinter 300. In particular, hydraulic system 125 includes injectors (not shown) to inject and extract fluid from the actuators of soft robotic arm 200 and 3D bioprinter 300 to control the movement of both arm 200 and bioprinter 300.

Computer system 1300 is in communication with hydraulic system 125 for actuating hydraulic system 125. The actuation includes actuating the injectors to inject and extract fluid. Computer system 1300 is described hereinafter in relation to FIGS. 11A and 11B below. Computer system 1300 has computer programs to transmit control signals to hydraulic system 125, which in turn actuates the injectors to inject/extract fluid from the actuators of arm 200 and bioprinter 300.

3D bioprinter 300 is mounted on the distal end of soft robotic arm 200. The distal end of soft robotic arm 200 is then propelled into an internal organ (e.g., intestine) of patient 150. As soft robotic arm 200 is being propelled into the organ, the distal end of soft robotic arm 200 is navigated by a user. The user is presented with a view of the organ (through which the arm 200 is being propelled) on a display screen of computer system 1300. The user then manipulates a user input (e.g., mouse, keyboard, haptic interface, etc.) to control the movement of the distal end of soft robotic arm 200.

In an alternative arrangement, other mechanisms (e.g., scalpel, etc.) may be mounted on the distal end of arm 200. The other mechanisms may also be mounted together with 3D bioprinter 300. The other mechanism may be mechanisms to perform endoscopy surgery.

Once soft robotic arm 200 reaches the surgical location within patient 150, the user navigates the distal end of soft robotic arm 200 to position the 3D bioprinter 300 over a target surface at the surgical location.

Similar to the operation of soft robotic arm 200, the user manipulates the user input of computer system 1300 to commence bioprinting. Computer system 1300 under the direction of a computer program then prints a 3D construct in vivo.

Hereinafter, the term “proximal end” refers to the end closer to hydraulic system 125, while the term “distal end” refers to the end situated away from hydraulic system 125.

FIG. 1B shows 3D bioprinter 300 mounted on the distal end of soft robotic arm 200.

FIG. 1C shows soft fibrous syringe architecture (SFSA) 175 which can be used as an alternative embodiment of the hydraulic system 125 for controlling the 3D printing head 300. The 3D printing head 300 can be controlled by soft artificial muscles being actuated by an electricity-free handheld device using the SFSA 175. The SFSA 175 connects two or more hydraulically actuated artificial muscles to function together, offering an electricity-free and motorless controlling method with sensing abilities for use in flexible robotics systems and wearable devices.

The SFSA 175 of FIG. 1C comprises a flexible hose 176 surrounded, portions of which are surrounded by portions of flexible artificial muscles 177a and 177b made of elastic tubes and helical coils. As shown in an enlargement, the flexible hose 176 can be a transmission tube 178 through which fluid (for example shown as water) can flow.

The portion of coil 177a can be stretched (see simulation examples 180 and 182) such that the portion of coil 177b is shortened (see simulation examples 181 and 183), or vice versa, to change the pressure of water in the hose 176. When the SFSA 175 is pressurised for two connected artificial muscles to pressure Pe, if one muscle (a master muscle, e.g. 177a) is stretched, the other muscle (a slave muscle, e.g. 177b) will be shortened as a working principle.

Using the SFSA 175, motion from controllers such as a joystick 190 or a wearable device 191 can be transferred to the soft robot arm 200 such that the 3D bioprinter 300 can deliver medical treatment. In the example of FIG. 1C examples 195 of hydraulic- or cable-driven robotic arms are shown. In an example 197 of medical treatment, the 3D bioprinter 300 can perform a 3D printing process.

### Soft Robotic Arm

### Structure of Soft Robotic Arm

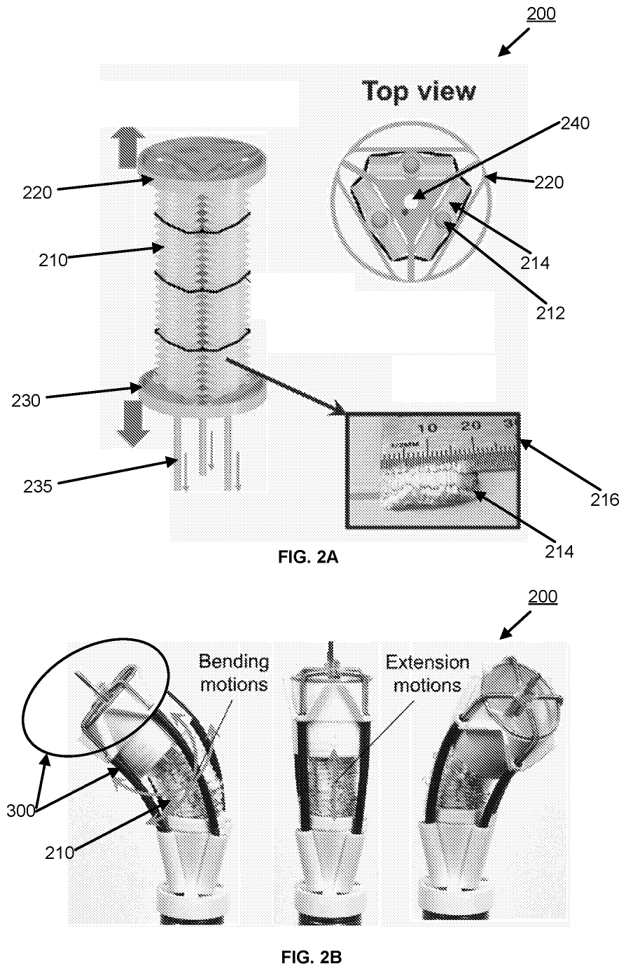

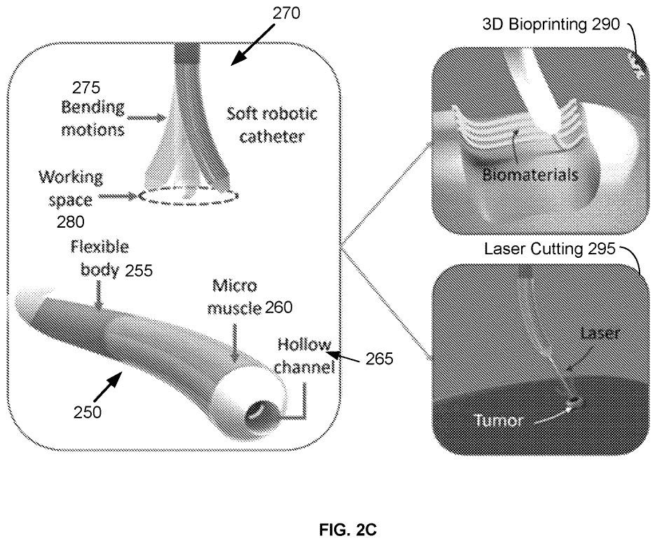

FIG. 2A shows the distal end of robotic arm 200. The distal end of robotic arm 200 is movable to navigate the arm 200 through an organ. FIG. 2B shows the bending and extension movements of the distal end of the robotic arm 200.

The distal end of robotic arm 200 includes actuators 210, proximal adapter 230, distal adapter 220, and fluid transmission tubes 235. The proximal ends of fluid transmission tubes 235 is in communication with hydraulic system 125, while the distal end of fluid transmission tubes 235 are in communication with actuators 210. Hydraulic system 125 injects/extracts fluid to and from the proximal end of fluid transmission tubes 235, which in turn increases or decreases the hydraulic pressure within fluid transmission tubes 235 and actuators 210.

Fluid transmission tubes 235 are covered by sheath 202 (see FIG. 1B), such that fluid transmission tubes 235 in conjunction with sheath 202 form the body of robotic arm 200. Fluid transmission tubes 235 are not stretchable. Examples of fluid transmission tubes 234 include PTFE tubes.

Each of actuators 210 has elastic tube 212 and means 214 for restricting the radial expansion of elastic tube 212. Means 214 include a conduit, within which elastic tube 212 is disposed. The proximal end of elastic tube 212 is connected to the distal end of fluid transmission tube 235 to receive the fluid from the hydraulic system 125. The distal end of elastic tube 212 is sealed. The proximal end of means 214 is connected to the proximal end of elastic tube 212, while the distal end of means 214 is connected to the distal end of elastic tube 212. Such connections enable means 214 to correspondingly lengthen and contract with tube 212 to maintain the radial expansion restriction of tube 212.

As hydraulic system 125 injects fluid into fluid transmission tube 235, the injected fluid increases the hydraulic pressure exerted onto elastic tube 212. Due to the increased hydraulic pressure, the distal end of tube 212 is the portion of tube 212 that is capable of stretching, as the radial expansion of tube 212 is restricted by means 214. Accordingly, the increased hydraulic pressure on tube 212 lengthens tube 212. As hydraulic system 125 extracts fluid from fluid transmission tube 235, the extracted fluid reduces hydraulic pressure from elastic tube 212, which in turn contracts tube 212.

In one arrangement, means 214 use non-stretchable fabric layers that are aligned and stacked along the warp and weft of the fabric layers. The fabric layers are then stitched together to form the conduit. In one arrangement, the conduit has a diameter of 3.5 mm. In one arrangement, any residual fabric is cut and heated (at 200° C.) along a cut edge 3 mm from the stitches to prevent fabric edges from fraying. The proximal end of the fabric layers is connected to the proximal end of elastic tube 212, while the distal end of the fabric layers is connected to the distal end of elastic tube 212. The fabric layers can also contract (via wrinkling, as shown in inset 216 of FIG. 2A) and lengthen with elastic tube 212.

The arrangement shown in FIG. 2A shows 3 actuators 210 being used. However, 4 or more actuators 210 may be used.

Each of the proximal ends of actuators 210 is attached to proximal adapter 230. Proximal adapter 230 is also used to connect bioprinter 300 to the distal end of robotic arm 200. Each of the distal ends of actuators 210 is attached to distal adapter 220. Actuators 210 are arranged in parallel to each other and the proximal and distal ends are disposed at the vertices of an equilateral triangle of adapter 220 and 230 (as shown on the top view of FIG. 2A), to enable omnidirectional motion of the distal end of robotic arm 200.

As described above, injecting and extracting fluid to and from elastic tubes 212 (via fluid transmission tubes 235) lengthens and contracts elastic tubes 212. When one of three actuators 210 is lengthened, the distal end of robotic arm 200 moves in one direction. When two actuators 210 are lengthened, then a bending movement in a plane between the two moved actuators 210 occurs. Similarly, when all three actuators 210 are lengthened equally, then a linear translation motion occurs along the axial axis of robotic arm 200.

As elastic tubes 212 are lengthened, elastic energy is stored in elastic tubes 212. While injecting fluid into elastic tubes 212 lengthens tubes 212, forces are typically generated when elastic tubes 212 contract (through fluid extraction by hydraulic system 125). Such a process is analogous to the human biological muscles.

Actuators 210 can be fabricated to a predetermined length. Further, the structure stiffness of actuators 210 can be tuned by using different types of elastic tubes and means 214.

The top view of FIG. 2A also shows distal adapter 220 having void 240. Void 240 is configured to receive camera 1327 (see FIG. 11A). Similarly, proximal adapter 230 also has a void to receive a cable of camera 1327. Camera 1327 and its cable are then disposed within sheath 202 so that the cable is in communication with computer system 1300 of control system 120. Computer system 1300 then provides a view captured by camera 1327 on video display 1314 (see FIG. 11A). A user controlling robotic arm 200 and bioprinter 300 can then view video display 1314 when manipulating robotic arm 200 via its input (e.g., haptic interface 1302).

FIG. 2C shows an alternative embodiment 250 of the soft robotic arm 200. In the example of FIG. 2C, the soft robotic arm 250 comprises at least a flexible body 255, a plurality of artificial micro muscles, for example micro muscle 260. A hollow channel 265 formed through the soft robotic arm 250 through which fluid can flow or light can travel. In the example of FIG. 2C, the soft robotic arm 250 comprises four artificial micro muscles arranged 90° around the hollow channel 265. The artificial micro muscles (such as 260) can be manufactured using known techniques, such as 3D printed silicon muscles for example. Once the inner pressures in the artificial micro muscles (such as 260) are different, the soft robotic arm 250 will bend. By contrast, if the pressures in the artificial micro muscles (such as 260) are equal, the soft robotic arm 250 will be lengthened or shortened compared to an original state. Accordingly, the soft robotic arm 250 can move in 3D space with three degrees of freedom. For example, a view 270 of a distal end of the robotic arm 250 shows bending motions 275 around a working space 280.

The hollow channel 265, also referred to as a central lumen, can be used to deliver materials for 3D bioprinting for medical treatment inside the human body. An example 290 of bioprinting is shown in FIG. 2C. Additionally, the hollow channel 265, can be used to transfer laser energy for cutting in minimally invasive surgery. An example 295 of laser cutting is shown in FIG. 2C.

### Method of Controlling Soft Robotic Arm

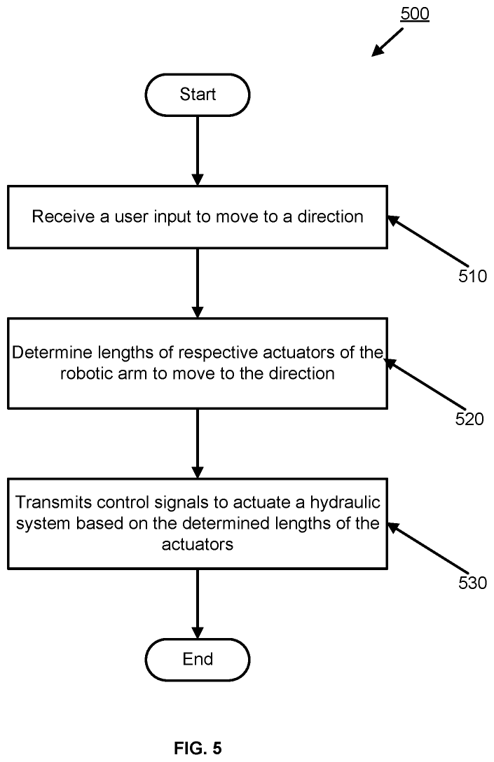

FIG. 5 shows method 500 of controlling soft robotic arm 200, in particular the distal end structure (as discussed above in relation to FIG. 2A) of arm 200. Method 500 is a computer program that is executable by computer system 1300 (described below in relation to FIGS. 11A and 11B.

Method 500 commences at step 510 by receiving a user input to move the distal end of robotic arm 200 to a direction. The user input may be received via haptic interface 1302, mouse pointer device 1303, and the like. If the SFSA 175 is used, the haptic interface 1302 may be a controller such as the joystick 190 or the wearable device 191, for example. The user is also provided with a video on video display 1314. The video is captured by camera 1327, which is situated at the distal end of robotic arm 200. By using the user input interface and video display 1314, the user is then capable of navigating robotic arm 200 through the organ.

Based on the user input, the distal end of robotic arm 200 moves accordingly. For example, the current direction of the distal end is toward the x direction on the xyz axes. The user moves the user input interface toward the z direction. Such a user input results in the distal end of robotic arm 200 moving in the corresponding direction from x direction to z direction.

Method 500 then proceeds from step 510 to step 520.

In step 520, computer system 1300 determines the lengths of respective actuators 210 of robotic arm 200 in order to move to the direction (as dictated by the user input received in step 510).

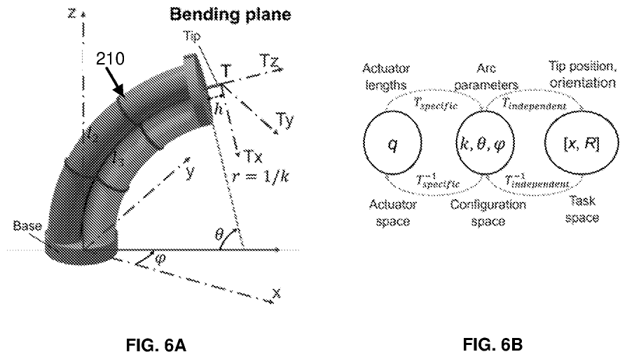

The relationship between lengths of actuators 210 with the distal end of robotic arm 200 is determined using a closed-form kinematic model. The closed-form kinematic model has two assumptions: all centre lines of actuators 210 are parallel at any working state due to mechanical constraints, and constant curvature is applied to all actuators 210 (see FIG. 6A).

FIG. 6B shows a kinematic model capturing the relationship between (1) the position and the orientation of the distal end of robotic arm 200 and (2) the length of each actuator 210. The kinematic model provides three working spaces: actuator space; configuration space; and task space. Actuator space q (l₁, l₂, l₃) provides the lengths of actuators 210. The actuator space is convertible to the configuration space C (k,θ,φ), which defines constant-curvature arcs of the distal end of robotic arm 200. The conversion from the actuator space to the configuration space uses a first homogenous transformation T_{specific} The configuration space is convertible to the task space [x,R] defining the position and orientation of the tip of the distal end of arm 200. The conversion from the configuration space to the task space uses a second homogenous transformation T_{independent}.

The three spaces are defined as follows:

$$x = {[ {T}_{x} , {T}_{y} , {T}_{z} ]}^{T} , R = {R}_{t} , R ∈ SO ( 3 ) \qquad ( 1 )$$

$$C = {[ k , θ , φ ]}^{T} \qquad ( 2 )$$

$$q = {[ {l}_{1} , {l}_{2} , {l}_{3} ]}^{T} \qquad ( 3 )$$

where x and R denote the position and orientation of the tip of the distal end of arm 200. k, θ and φ represent the arc parameters of robotic arm 200 where k represents the curvature, θ is the rotational angle about the y-axis compared to the arm pose when all actuators 210 are at rest and φ is the bending direction angle of the plane containing the arc compared to +x-axis. l₁, l₂ and l₃ denote the lengths of each of actuators 210 at its centerline.

A closed-form relationship between the lengths of actuators 210 and the configuration space parameters is expressed as follows:

$$l = \frac{{l}_{1} + {l}_{2} + {l}_{3}}{3} \qquad ( 4 )$$

$$k = \frac{2 \sqrt{{l}_{1}^{2} + {l}_{2}^{2} + {l}_{3}^{2} - {l}_{1} {l}_{2} - {l}_{1} {l}_{3} - {l}_{2} {l}_{3}}}{d ( {l}_{1} + {l}_{2} + {l}_{3} )} \qquad ( 5 )$$

$$θ = kl \qquad ( 6 )$$

$$φ = {tan}^{- 1} ( \frac{\sqrt{3} ( {l}_{2} + {l}_{3} - 2 {l}_{1} )}{3 ( {l}_{2} - {l}_{3} )} ) \qquad ( 7 )$$

Where l represents the current length of the imaginary central backbone of actuators 210 and d is the distance from the centre of the imaginary central backbone to the centre of actuator 210, which is the same for all actuators 210. From Eq. (7) the bending direction angle φ is derived based on the length of each actuator 210.

As indicated above, the actuator space is converted to the configuration space using a first homogenous transformation T_{specific}, where the equation is: C=T_{specific}. T_{specific} is derived from the equations (4) to (7).

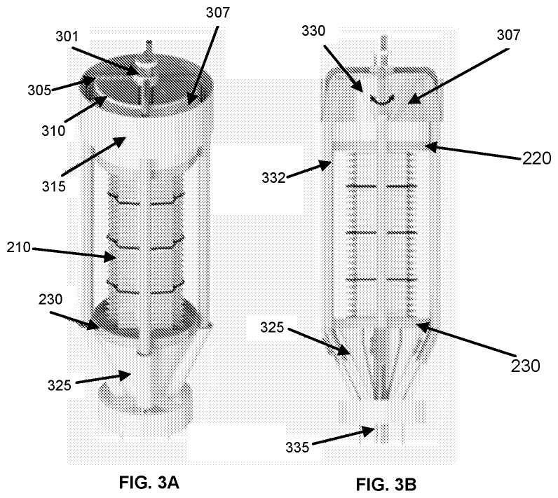

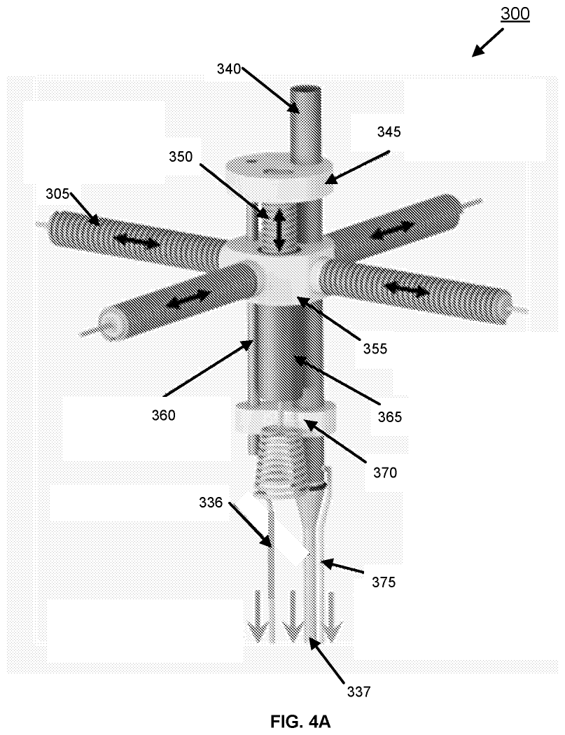

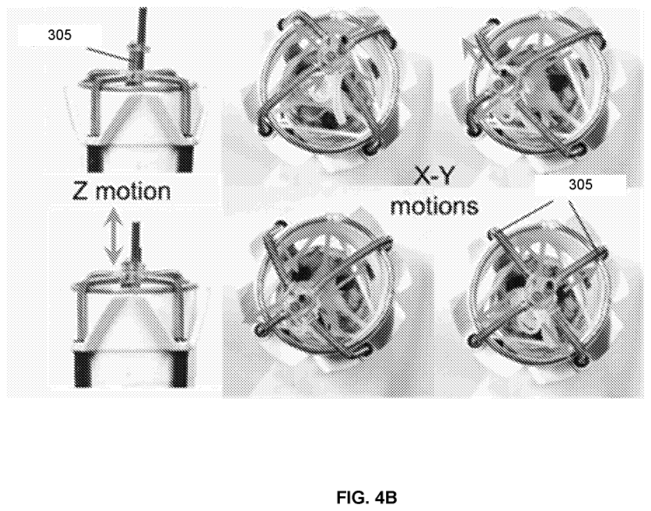

The tip position vector x and rotation matrix R for the tip of the distal end of arm 200 are calculated by modifying the second homogeneous transformation (T_{independent}) expressed in Eqs. (7) and (8) as:

$$( 8 )$$

$${T}_{independent} = [ \begin{matrix}cos φ * cosks & - sin φ & cos φ * sinks & - cos φ * ( \frac{cosks - 1}{k} - h * sinks ) \\ sin φ * cosks & cos φ & sin φ * sinks & - sin φ * ( \frac{cosks - 1}{k} - h * sinks \\ - sinks & 0 & c o s k s & h * cosks + \frac{sinks}{k} \\ 0 & 0 & 0 & 1\end{matrix} ]$$

where s∈[0 l] and h is the length of the arm tip (i.e., the combination of the height of top platform 345 in relation to middle platform 355 and print nozzle 340—see description below in relation to FIGS. 3A, 3B, and 4).

For example, the tip position vector x=0 mm and rotation matrix R=0° at the current position. A user input is then received at step 510 to move the tip position vector x=90 mm and rotation matrix R=90°. At step 520, computer system 1300 uses equations 4 to 8 to determine the lengths 11, 12 and 3 of respective actuators 210 to obtain the tip position vector x=90 and rotation matrix R=90.

Method 500 then proceeds from step 520 to step 530.

In step 530, computer system 1300 transmits control signals to actuate hydraulic system 125 to inject or extract fluid from fluid transmission tubes 235 in order to lengthen or shorten actuators 210 according to the determined lengths l₁, l₂ and l₃ (which are calculated at step 520).

The relationship between hydraulic pressure within fluid transmission tubes 235 and actuators 210 is dependent on the elastic material used on tube 212. Such a relationship is determined experimentally. Further, hydraulic system 125 includes a pressure sensor to determine the hydraulic pressure of each actuator 210.

Method 500 concludes at the conclusion of step 530 and is repeated whenever the distal end of arm 200 needs to be moved.

### 3D Bioprinter

### Structure of 3D Bioprinter

FIGS. 3A and 3B show 3D bioprinter 300. FIG. 3A is a perspective view of 3D bioprinter 300, while FIG. 3B is a side view of 3D bioprinter 300. FIG. 4A shows a close-up view of printing mechanism 301 of 3D bioprinter 300. FIG. 4B shows movements of printing mechanism 301 of 3D bioprinter 300.

3D bioprinter 300 has printing mechanism 301, soft microtubule artificial muscles (SMAMs) 305, printing guide 310, protective cover 315, guide 325, and fluid transmission tubes 335.

Printing guide 310 is a cylindrical tube where the distal end is a printing area over which printing mechanism 301 can move, while the proximal end is an adapter to connect with distal adapter 220. Printing guide 310 can be in the shape of a ring (as shown in FIG. 3A), a rectangle, an oval, and the like. In another arrangement, printing guide 310 can be separated into different components, where one component provides the printing area and another component is the adapter to connect with distal adapter 220.

Printing mechanism 301 is disposed at the printing area of printing guide 310. FIG. 4 shows the printing mechanism 301 in more details. Printing mechanism 301 includes print nozzle 340; a body having top platform 345, middle platform 355, and bottom platform 370; SMAM 350, guide rod 360, and protective sheath 365. Printing mechanism 301 may be replaced with other mechanisms such as a gripping mechanism, a cutting mechanism, and the like, so that other functions may be performed.

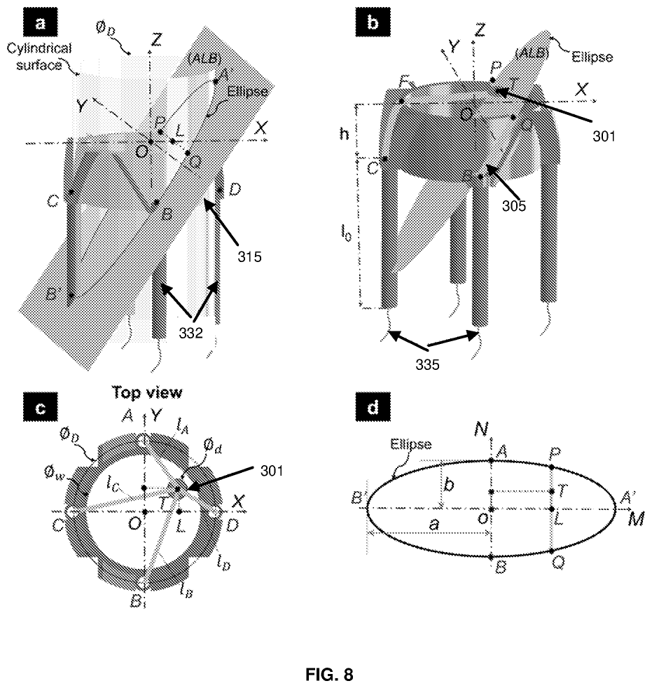

Print nozzle 340 is disposed on top platform 345 and extends through middle platform 355 to bottom platform 370. Top platform 345 is movable in relation to middle platform 355 to enable printing in the z direction (see FIG. 8). Accordingly, print nozzle 340 is retractable to allow movement of top platform 345 in relation to middle platform 355.

Top platform 345 is connected to middle platform 355 and bottom platform 370 via guide rod 360. Middle platform 355 is disposed on the printing area of printing guide 310 and is movable on the printing area. Accordingly, components of printing mechanism 301 below middle platform 355 is disposed within the cylindrical tube of printing guide 310. Guide rod 360 is fixedly connected to middle platform 355 and bottom platform 370. Guide rod 360 is movably connected to top platform 345, to enable top platform 345 to be movable in relation to middle platform 355.

SMAM 350 is fixedly connected to top platform 345 to move top platform 345 in relation to middle platform 355. The operation of the SMAM is described in PCT/AU2021/050922 and PCT/AU2021/050924, the contents of which are incorporated herein by reference in their entirety. SMAM 350 is the actuator of 3D bioprinter 300.

When SMAM 350 is hydraulically pressurized by hydraulic system 125, SMAM 350 pushes top platform 345 upward (i.e., away from middle platform 355). Guide rod 310 constrains the movement of top platform 350 to the perpendicular direction (i.e., z direction-see FIG. 8) of the XY plane (see FIG. 8). When the hydraulic pressure drops, the contraction force of SMAM 350 brings top platform 345 downward (i.e., toward middle platform 355). SMAM 350 is extendible by up to 6 mm and can exert an axial force of 1.5 N. Accordingly, SMAM 350 actuates print nozzle 340 longitudinally. Fluid transmission tube 336 provides the hydraulic pressure to SMAM 350. Fluid transmission tube 336 is connected to SMAM 350 via the voids of proximal adapters 220 and 230. Fluid transmission tube 336 is protected by protective sheath 365.

Fluid transmission tube 337 is connected to print nozzle 340 to provide the ink to print nozzle 340. Print nozzle 340 includes a valve within the nozzle to control the amount of ink ejected by print nozzle 340. Communication cable 375, which is connected to computer system 1300, provides control signals to the valve of print nozzle 340.

Middle platform 355 is also movable on the XY plane over the printing area of printing guide 310. To do so, SMAMs 305 are fixedly connected to middle platform 355. By exerting force over one or more SMAMs 305, middle platform 355 is movable to different locations of the printing area of printing guide 310. Although four SMAMs 305 are shown, it is possible to use three, five, or more SMAMs 305 to move middle platform 345 over the printing area. The movement of middle platform 355 in turn moves printing mechanism 301 over the XY plane.

In one arrangement, the four SMAMs 305 shown in FIG. 3A are arranged in a cross shape. The ring shape of printing guide 310 has an inner diameter of 17 mm. Each of SMAMs 305 is configured to extend by a length of 30 mm over the printing area, so that printing mechanism 301 is movable over the printing area of printing guide 310.

Fluid transmission tubes 335 provide controls over SMAMs 305, similar as to how fluid transmission tubes 235 provide controls over elastic tubes 212.

Protective cover 315 is a cylindrical tube protecting printing guide 310 as shown in FIG. 3A. Protective cover 315 protects printing guide 310 and SMAMs 305 as robotic arm 200 navigates through an organ. Protective cover 315 is connected to printing guide 310.

In the arrangement shown in FIGS. 3A and 3B, protective guide 315 has a proximal end and a distal end. The distal end of protective guide 315 is aligned with the printing area of printing guide 310. The proximal end of protective guide 315 extends below the printing area of printing guide 310.

The proximal end of protective guide 315 connects to the edges of the printing area of printing guide 310. Such connection provides a taper over which each of SMAMs 305 is disposed. The taper provides a gradual transition for SMAMs 305. The proximal end of protective cover 315 provides voids into which respective SMAMs 305 goes through. Each SMAM 305 is then disposed over the taper and over the edges of the printing area of printing guide 310. Such an arrangement enables SMAMs 305 to be anchored at a particular location at the proximal end of protective cover 315. The taper between protective cover 315 and the printing area of printing guide 310 then prevents each of SMAMs 305 from having a 90° bend (which may result in movement issues), while providing the necessary movement of printing mechanism 301 over the printing area. Further, to avoid one SMAM 305 from contacting another SMAM 305, borders 307 are disposed over the taper area between protective cover 315 and printing guide 310.

In the arrangement shown in FIG. 3B, the combination of the taper between protective cover 315 and printing guide 310 and borders 307 define a V-shape enclosed space 330 in which respective SMAMs 305 can move, when SMAMs 305 are moving printing mechanism 301.

Each of SMAMs 305 then goes over the edge of the printing area of printing guide 310. The edges of the printing area of printing guide 310 are connected to the proximal end of protective cover 315.

In one arrangement, a low friction material is disposed on the edges of the printing area of printing guide 310 to reduce the friction between SMAMs 305 and the edges.

Each void provided at the proximal end of protective cover 315 has protective sheath 332 extending from the void toward the proximal adapter 230. Respective SMAMs 305 are disposed within protective sheath 332. The other end of protective sheath 332 is connected to guide 325. Guide 325 is connected to proximal adapter 230.

Guide 325 provides guiding means for SMAMs 305 around the structure of the distal end of robotic arm 200. SMAMs 305 are guided by guide 325 to a central area in which each of SMAMs 305 is connected to fluid transmission tubes 335. Fluid transmission tubes 235 (controlling actuators 210 of the distal end of robotic arm 200) are disposed within guide 325, to allow tubes 235 to connect to actuators 210.

### Method of Controlling 3D Bioprinter

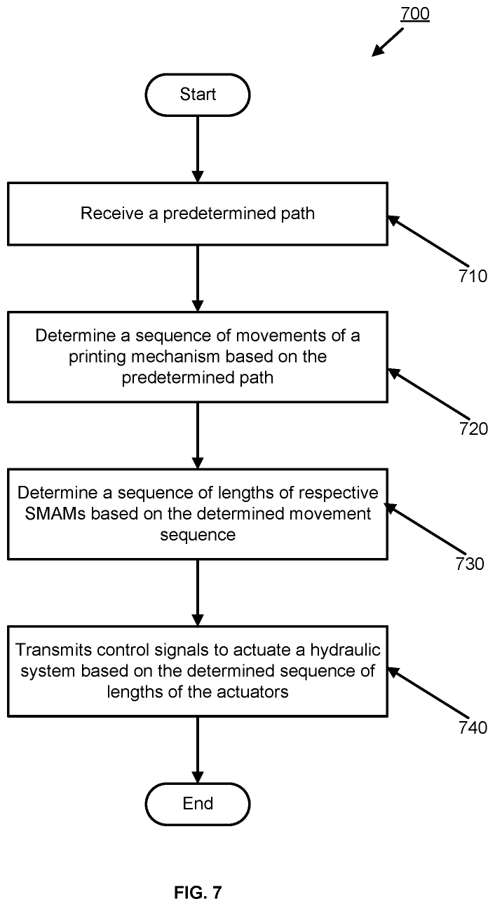

FIG. 7 shows method 700 of controlling 3D bioprinter 300. Method 700 is a computer program that is executable by computer system 1300 (described below in relation to FIGS. 11A and 11B.

Method 700 commences at step 710 by receiving a predetermined path over which print nozzle 340 is to traverse. The predetermined path is determined by a user via computer system 1300. For example, a user provides the predetermined path via mouse pointer device 1303, and the like.

Method 700 then proceeds from step 710 to step 720.

In step 720, computer system 1300 determines a sequence of movements of printing mechanism 301 based on the predetermined path.

For example, if the predetermined path is a rectangle, computer system 1300 determines that the sequence of movements to achieve the rectangle is a horizontal right movement from first coordinate points to second coordinate points, a vertical down movement from the second coordinate points to third coordinate points, a horizontal left movement from the third coordinate points to fourth coordinate points, and a vertical up movement from the fourth coordinate points to the first coordinate points.

Method 700 then proceeds from step 720 to step 730.

In step 730, computer system 1300 determines a sequence of lengths of respective SMAMs 305 of printing mechanism 301 based on the determined sequence of movement of printing mechanism 301.

The sequence of lengths of respective SMAMs 305 is determined based on an inverse kinematic model representing a relationship between the position T(X,Y) of print nozzle 340 and the length (l_{A},l_{B},l_{C},l_{D}) of each SMAM 305. Method 700 is discussed in relation to using four SMAMs 305, but a skilled person would understand that the inverse kinematic model is adaptable for use by different numbers of SMAMs 305.

The development of the inverse kinematic model of SMAMs 305 is now described.

Each SMAM 305 is initially pressurized to reach a certain length that is longer than a nominal length of SMAM 305 (i.e., the length at zero hydraulic pressure). FIGS. 8a to 8d show the schematic diagrams for 3D bioprinter 300, where A, B, C, and D are the locations of the voids at the proximal end of protective cover 315 and O is the central point of printing guide 310 with a diameter Ø_{d} (when the SMAMs 305 are at the resting state).

The plane ALB (see FIGS. 8a and 8d) passes through three points (L(X,0) in XOY-coordinate, A, and B) and intersects with a circle of diameter Øp containing four points A, B, C, and D to form an ellipse (see FIG. 8d) with a central point o(0,0). The points P, T, and Q are collinear, lying on a single line which is the intersection between the plane XOY and the plane ALB. The point o(0,0) is the central point of the line through A and B. The semi-major axis with a length of a and the semi-minor axis with a length of b of the ellipse can be determined by:

$$\{ \begin{matrix}a = \sqrt{\frac{{P}_{M}^{2}}{1 - \frac{{P}_{N}^{2}}{{b}^{2}}}} \\ b = \frac{{∅}_{D}}{2}\end{matrix} with \{ \begin{matrix}{P}_{M} = oL = \sqrt{{OL}^{2} + {h}^{2}} = \sqrt{{X}^{2} + {h}^{2}} \\ {P}_{N} = PL = \sqrt{{OL}^{2} - {OL}^{2}} = \sqrt{\frac{{∅}_{D}}{2} - {X}^{2}} \\ X < \frac{{∅}_{D}}{2} , Y < \frac{{∅}_{D}}{2}\end{matrix} \qquad ( 9 )$$

where P_{M}, P_{N} are the projection of P on the M-axis and N-axis of the plane MON, respectively; h is the distance between the plane (ABC) and the plane (XOY).

The entire length of two opposite SMAMs (l_{A},l_{B}) passing through two points A and B (see FIGS. 8b and 8c) can be calculated by:

$$\{ \begin{matrix}{l}_{A} = {l}_{0} + AP + PT - 0.5 {∅}_{d} = {l}_{0} + AP + {P}_{N} - Y - 0.5 {∅}_{d} \\ {l}_{B} = {l}_{0} + BQ + QT - 0.5 {∅}_{d} = {l}_{0} + BQ + {P}_{N} + Y - 0.5 {∅}_{d}\end{matrix} \qquad ( 10 )$$

$$\begin{matrix}AP = BQ = {\int }_{0}^{{P}_{M}} \sqrt{1 + {( \frac{dN}{dM} )}^{2}} dM \\ = {\int }_{0}^{{P}_{M}} \sqrt{1 - \frac{4 {M}^{2} {X}^{4}}{{∅}_{D}^{2} ( \frac{4 {M}^{2} {X}^{2}}{{∅}_{D}^{2} ( {h}^{2} + {X}^{2} )} - 1 ) {( {h}^{2} + {X}^{2} )}^{2}}} dM\end{matrix} \qquad ( 11 )$$

where l₀ is the length of SMAM 305 inside protective sheath 332.

Similarly, the entire length (l_{C},l_{B}) of the other SMAMs 305 is expressed by:

$$\{ \begin{matrix}{l}_{C} = {l}_{0} + CF + {F}_{M} + X - 0.5 {∅}_{d} \\ {l}_{D} = {l}_{0} + CF + {F}_{M} - X - 0.5 {∅}_{d}\end{matrix} \qquad ( 12 )$$

$$( 13 )$$

$$\{ \begin{matrix}{F}_{M} = \sqrt{{( 0.5 {∅}_{D} )}^{2} - {Y}^{2}} \\ {F}_{N} = \sqrt{{Y}^{2} + {h}^{2}} \\ CF = {\int }_{{F}_{M}}^{0.5 {∅}_{D}} \sqrt{1 + {( \frac{dN}{dM} )}^{2}} dM = {\int }_{{F}_{M}}^{0.5 {∅}_{D}} \sqrt{1 - \frac{4 {M}^{2} ( {Y}^{2} + {h}^{2} )}{{∅}_{D}^{2} {Y}^{2} ( \frac{4 {M}^{2}}{{∅}_{D}^{2}} - 1 )}} dM\end{matrix}$$

Using the above discussed inverse kinematic model and the example in step 720, computer system 1300 determines the necessary lengths of respective SMAMs 305 to move from the first coordinate points to the second coordinate points for the right horizontal movement. Computer system 1300 also determines the necessary lengths of respective SMAMs 305 for the other movements (e.g., vertical down movement, etc.).

Method 700 then proceeds from step 730 to step 740.

In step 740, computer system 1300 transmits control signals to actuate hydraulic system 125 based on the determined sequence of lengths of SMAMs 305. The actuation of hydraulic system 125 includes injecting and extracting fluid from fluid transmission tubes 335, which in turn lengthens and shortens SMAMs 305 correspondingly. However, there is hysteresis between fluid displacement by an injector of hydraulic system 125 (i.e., input) and SMAM 305 changing length (i.e., output). Further, the hysteresis profile of SMAM 305 is asymmetric, meaning the hysteresis at the loading phase (i.e., when fluid is injected into SMAM 305) is different to the unloading phase (i.e., when fluid is extracted from SMAM 305).

The asymmetric hysteresis profile is due to memory effects, nonlinear friction, and elastic deformation of soft materials. In turn, the asymmetric hysteresis profile means that the length of SMAM 305 is highly dependent on the current state (i.e., motion and hydraulic pressure) of SMAM 305. Therefore, to accurately actuate hydraulic system 125, a hysteresis profile of SMAM 305 needs to be determined and developed. The hysteresis model of SMAM 305 to be described offers a fewer number of model parameters, higher accuracy, and less computational time in comparison to conventional hysteresis models.

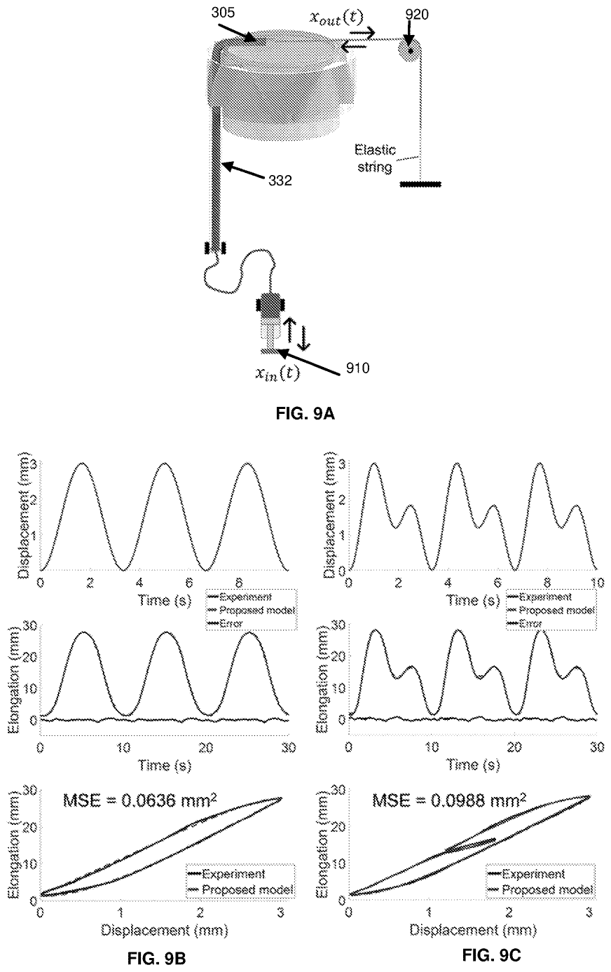

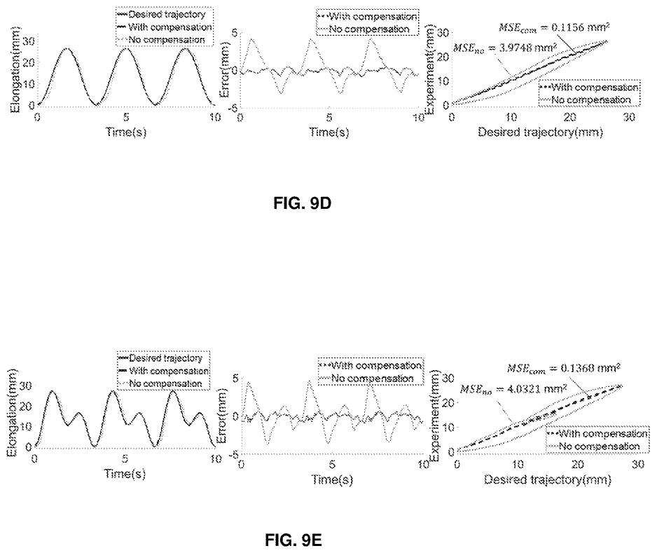

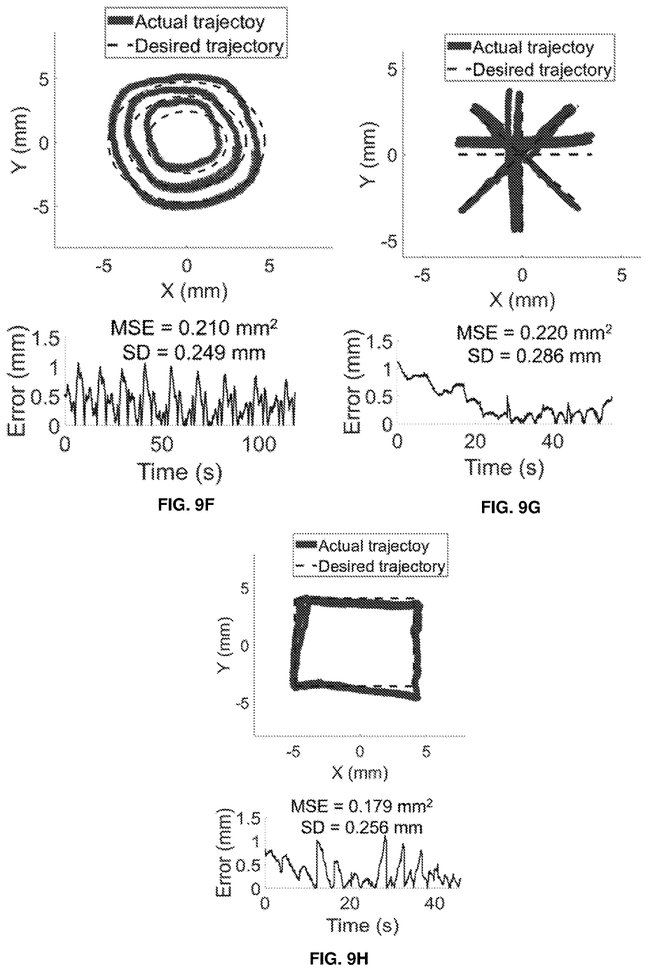

To determine the hysteresis profile of SMAM 305, SMAM 305 is disposed in printing guide 310. The distal end of SMAM 305 is connected to an elastic string with a load at the distal end of the elastic string. Further, encoder 920 is used to measure the elongation of SMAM 305. The displacement of injector 910 of hydraulic system 125 is called input displacement x(t)=xᵢₙ(t) (see FIG. 9A). The elongation of SMAM 305 is called output elongation Φ_{S}(x,t)=xₒᵤₜ(t).

The asymmetric hysteresis model of SMAM 305 is shown in FIGS. 9B and 9C. FIG. 9B shows three diagrams. The top diagram of FIG. 9B shows the input displacement over time (i.e., about 10 seconds). The middle diagram of FIG. 9B shows the output elongation over time (i.e., about 30 seconds), where the output elongation lags behind the input displacement shown in the top diagram. The bottom diagram of FIG. 9B shows a comparison of the input displacement versus the output elongation. The bottom diagram of FIG. 9B shows the asymmetric hysteresis loop of SMAM 305. The loading phase is the upper line of the hysteresis loop, while the unloading phase is the bottom line of the hysteresis loop.

FIG. 9C shows a hysteresis loop when a different pattern of input displacement (see the top diagram of FIG. 9C) is used.

From experiments, the hysteresis model is expressed by:

$${Φ}_{s} ( x , t ) = \{ \begin{matrix}0 \text{:} x ( t ) < 0 \\ {α}_{x 1} tan h ( x ( t ) / {α}_{x 2} ) + {α}_{z} z ( t ) \text{:} x ( t ) \geq 0\end{matrix} \qquad ( 14 )$$

$$\dot{z} ( t ) = \vert \dot{x} ( t ) \vert [ A sgn ( \dot{x} ( t ) ) - v {\vert z ( t ) \vert}^{n - 1} z ( t ) + ρ ] \qquad ( 15 )$$

Where z(t) is the internal state of the hysteresis model which represents the average deformation of elastic materials used in SMAMs 305. A hyperbolic tangent is incorporated into the hysteresis model to smoothen the reverse curve at the transition point of the hysteresis loop. The dimensionless parameters A, v, ρ, and n in eqns. (14) and Eq. (15) control the shape and size of the hysteresis loop. The coefficients αₓ₁, αₓ₂, and α_{z} represent the ratio of output elongation Φ_{S}(x,t) to the input displacement x(t) and the internal state z(t).

By minimizing the mean square error (MSE) between the outputs of the hysteresis model and the measured experimental data based on Particle Swarm Optimization (PSO), seven parameters are identified and optimized. The identified model parameters using PSO are αₓ₁=38.81, αₓ₂=3.20, α_{z}=−0.13, A=34.54, v=1.01, ρ=4.43 and n=1.05. FIGS. 9B and 9C show that the parameters used in the hysteresis model in eqns. (14) and Eq. (15) correlate to the experimental data.

The hysteresis model identified in eqns. (14) and (15) is then inverted to compensate for the hysteresis loop when actuating hydraulic system 125.

When implementing the compensation, the initial length and fluid pressure for SMAMs 305 are kept the same for the hysteresis determination process (see FIG. 9A) and during normal operation (see FIG. 3A). FIG. 9D shows the output elongation using the input displacement pattern of FIG. 9B. FIG. 9E shows the output elongation using the input displacement pattern of FIG. 9C.

The middle diagrams of FIGS. 9D and 9E show that there is a higher tracking error between the desired trajectory x_{d}(t) and the measured output elongation xₒᵤₜ(t) if the compensation is not implemented.

Quantitatively, the tracking performance for the case of a single frequency (0.3 Hz) had an MSEₙₒ=3.9748 mm² (without compensation) and MSE_{com}=0.1156 mm² (with compensation). For the periodic reference (combination of 0.3 Hz and 0.6 Hz), the MSEₙₒ=4.0321 mm² without compensation and MSE_{com}=0.1368 mm² if the compensation is engaged.

Using the inverse of the hysteresis model of eqns. (14) and (15), computer system 1300 transmits control signals to hydraulic system 125 that compensates for the asymmetric hysteresis profile.

FIGS. 9F to 9H show the performance of 3D bioprinter 300 when computer system 1300 uses the compensation. The actual path of 3D bioprinter 300 closely matches the predetermined path.

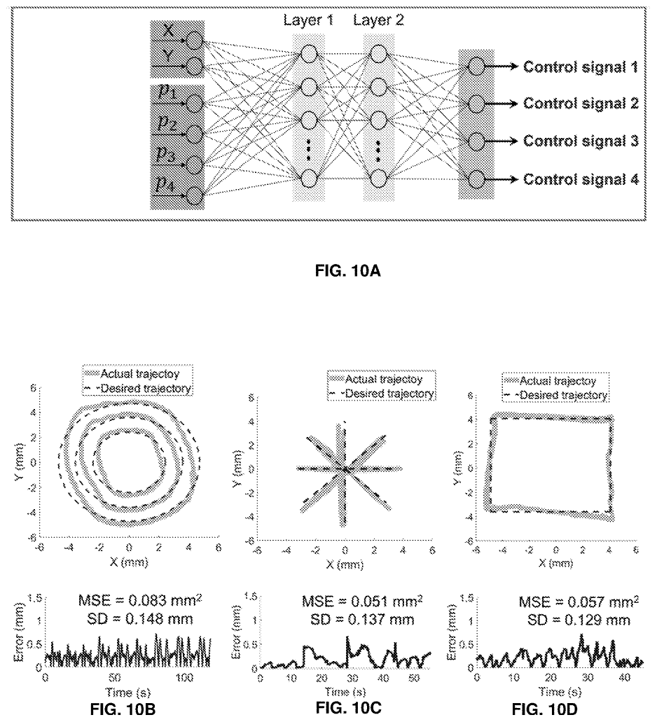

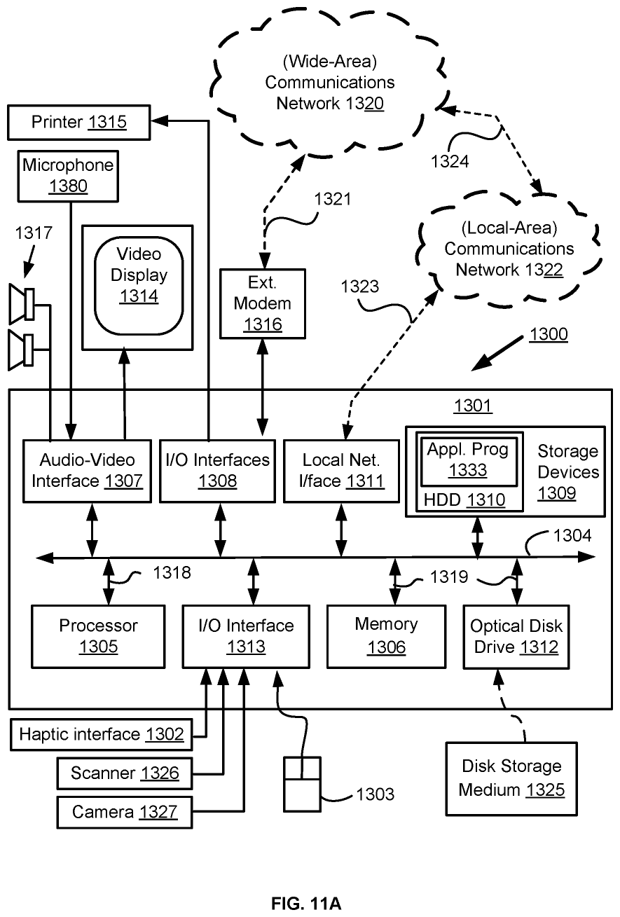

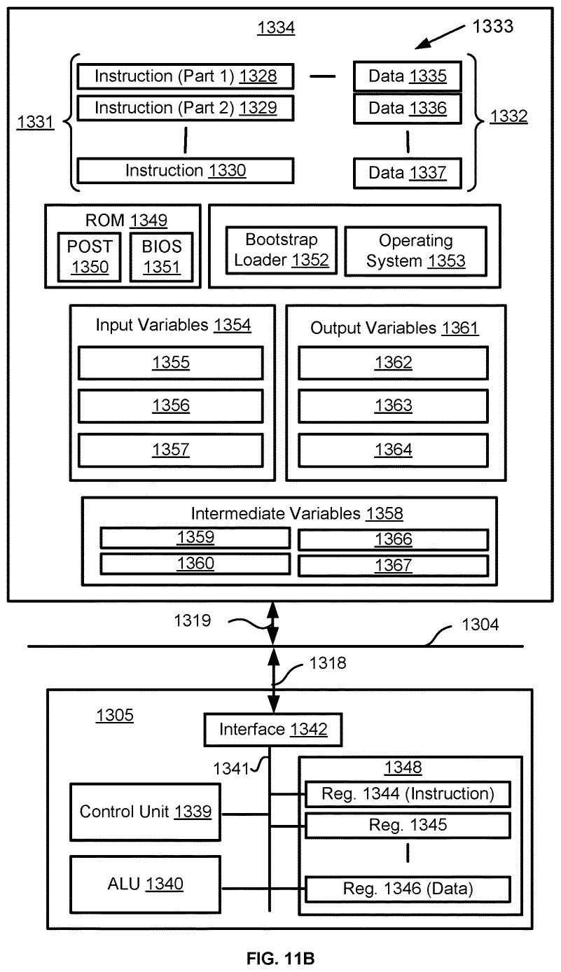

In an alternative arrangement, method 700 deploys a neural network (e.g., MATLAB's Dynamic Time-series function) to control the actuation of hydraulic system 125. An example of the neural network is shown in FIG. 10A.

The neural network receives inputs of time-varying 2D position (X, Y) of print nozzle 340 and pressures (p₁,p₂,p₃,p₄) of SMAMs 305. The neural network then outputs four control signals for the respective injectors of hydraulic system 125. Each injector controls one SMAM 305.

The neural network needs to be trained first using datasets. To create the datasets, print nozzle 340 is controlled to follow various patterns such as concentric circles, rectangles, and spirals with a sampling rate of 100 Hz. The movement of print nozzle 340 is verified manually. The developed inverse kinematic model given by eqns. (9) to (13) is used to calculate the required input displacement of the injectors from linear motion units.

One thousand datasets are collected and randomly split into training/validation/testing sets in the ratio of 75/15/15, respectively. A neural network with two hidden layers and Bayesian regularization backpropagation is used to train the network parameters. The performance of the trained neural network is shown in FIGS. 10B to 10D, where it can be seen that the paths of print nozzle 340 closely follows the predetermined paths.

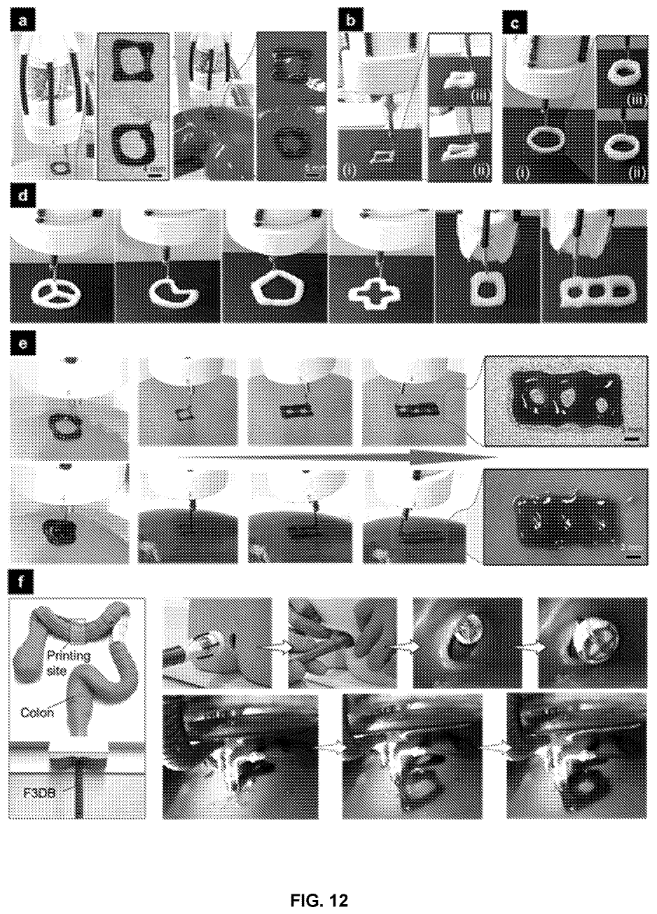

FIG. 12 shows the printing performance of a prototype of 3D bioprinter 300 mounted on arm 200. FIG. 12(a) shows printing of liquid chocolate with three-layered rectangular and circular shapes on a flat surface (left panel) and fresh porcine kidney (right panel). (b-c) 3D printing of gel composite made from cationic polymers, silicones, alcohol, and olive oil on a flat surface with three layers (i), five layers (ii), and seven layers (iii). (d) 3D printing of gel composite on a flat surface with different shapes and layers. (e) Circular printing of liquid chocolate on a flat surface with concentric filling (left panel) and multisite printing of liquid chocolate (right panel) on a flat surface (top figures) and a fresh porcine kidney (bottom figures). (f) In situ 3D printing of two-layered liquid chocolate inside a colon phantom (transverse colon segment). (Top right panel) Insertion process of arm 200 and 3D bioprinter 300 into the colon channel to reach the target site within the transverse colon segment via the anal canal and rectum. (Bottom right panel) In situ printing process of the two-layered rectangular shape onto the inner surface of the colon phantom.

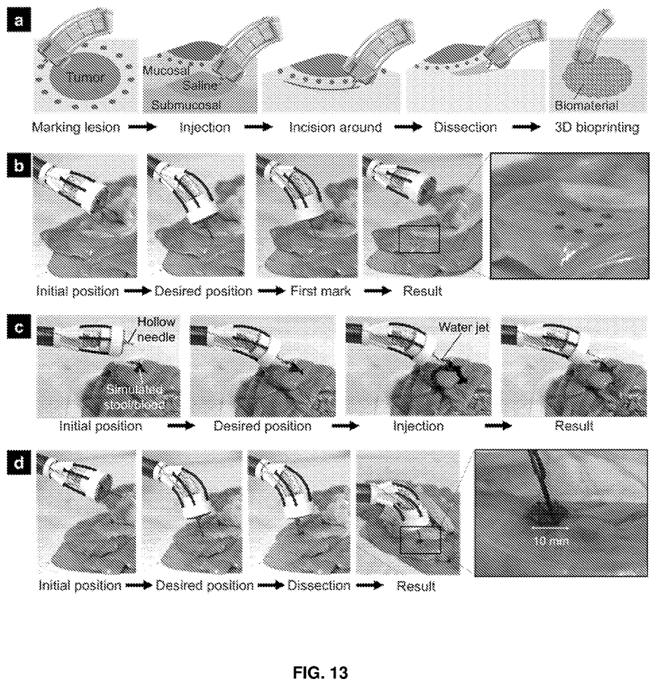

FIG. 13 shows an endoscopic surgery using soft robotic arm 200 and 3D bioprinter 300. FIG. 13(a) shows an illustration of endoscopic submucosal dissection (ESD) procedure with arm 200 and 3D bioprinter 300. (b) Demonstration of marking lesion perimeter with electrosurgery. (c) Demonstration of washing lesion surface. (d) Demonstration of the circular dissection in a lesion.

### Computer Description

FIGS. 11A and 11B depict a general-purpose computer system 1300, upon which the various arrangements described can be practiced.

As seen in FIG. 11A, the computer system 1300 includes: a computer module 1301; input devices such as a haptic interface 1302, a mouse pointer device 1303, a scanner 1326, a camera 1327, and a microphone 1380; and output devices including a printer 1315, a display device 1314 and loudspeakers 1317. An external Modulator-Demodulator (Modem) transceiver device 1316 may be used by the computer module 1301 for communicating to and from a communications network 1320 via a connection 1321. The communications network 1320 may be a wide-area network (WAN), such as the Internet, a cellular telecommunications network, or a private WAN. Where the connection 1321 is a telephone line, the modem 1316 may be a traditional “dial-up” modem. Alternatively, where the connection 1321 is a high capacity (e.g., cable) connection, the modem 1316 may be a broadband modem. A wireless modem may also be used for wireless connection to the communications network 1320.

The computer module 1301 typically includes at least one processor unit 1305, and a memory unit 1306. For example, the memory unit 1306 may have semiconductor random access memory (RAM) and semiconductor read only memory (ROM). The computer module 1301 also includes an number of input/output (I/O) interfaces including: an audio-video interface 1307 that couples to the video display 1314, loudspeakers 1317 and microphone 1380; an I/O interface 1313 that couples to the haptic interface 1302, mouse 1303, scanner 1326, camera 1327 and optionally a joystick or other human interface device (not illustrated); and an interface 1308 for the external modem 1316 and printer 1315. In some implementations, the modem 1316 may be incorporated within the computer module 1301, for example within the interface 1308. The computer module 1301 also has a local network interface 1311, which permits coupling of the computer system 1300 via a connection 1323 to a local-area communications network 1322, known as a Local Area Network (LAN). As illustrated in FIG. 11A, the local communications network 1322 may also couple to the wide network 1320 via a connection 1324, which would typically include a so-called “firewall” device or device of similar functionality. The local network interface 1311 may comprise an Ethernet circuit card, a Bluetooth® wireless arrangement or an IEEE 802.11 wireless arrangement; however, numerous other types of interfaces may be practiced for the interface 1311.

The I/O interfaces 1308 and 1313 may afford either or both of serial and parallel connectivity, the former typically being implemented according to the Universal Serial Bus (USB) standards and having corresponding USB connectors (not illustrated). Storage devices 1309 are provided and typically include a hard disk drive (HDD) 1310. Other storage devices such as a floppy disk drive and a magnetic tape drive (not illustrated) may also be used. An optical disk drive 1312 is typically provided to act as a non-volatile source of data. Portable memory devices, such optical disks (e.g., CD-ROM, DVD, Blu-ray Disc™), USB-RAM, portable, external hard drives, and floppy disks, for example, may be used as appropriate sources of data to the system 1300.

In one arrangement, the computer module 1301 also includes analog input/output ports (not shown) for receiving analog inputs and outputs.

The components 1305 to 1313 of the computer module 1301 typically communicate via an interconnected bus 1304 and in a manner that results in a conventional mode of operation of the computer system 1300 known to those in the relevant art. For example, the processor 1305 is coupled to the system bus 1304 using a connection 1318. Likewise, the memory 1306 and optical disk drive 1312 are coupled to the system bus 1304 by connections 1319. Examples of computers on which the described arrangements can be practised include IBM-PC's and compatibles, Sun Sparcstations, Apple Mac™ or like computer systems.

The method of controlling robotic arm 200 and 3D bioprinter 300 may be implemented using the computer system 1300 wherein the processes of FIGS. 5 and 7, described above, may be implemented as one or more software application programs 1333 executable within the computer system 1300. In particular, the steps of the method of controlling robotic arm 200 and 3D bioprinter 300 are effected by instructions 1331 (see FIG. 11B) in the software 1333 that are carried out within the computer system 1300. The software instructions 1331 may be formed as one or more code modules, each for performing one or more particular tasks. The software may also be divided into two separate parts, in which a first part and the corresponding code modules performs the control methods and a second part and the corresponding code modules manage a user interface between the first part and the user.

The software may be stored in a computer readable medium, including the storage devices described below, for example. The software is loaded into the computer system 1300 from the computer readable medium, and then executed by the computer system 1300. A computer readable medium having such software or computer program recorded on the computer readable medium is a computer program product. The use of the computer program product in the computer system 1300 preferably effects an advantageous apparatus for in vivo bioprinting.

The software 1333 is typically stored in the HDD 1310 or the memory 1306. The software is loaded into the computer system 1300 from a computer readable medium, and executed by the computer system 1300. Thus, for example, the software 1333 may be stored on an optically readable disk storage medium (e.g., CD-ROM) 1325 that is read by the optical disk drive 1312. A computer readable medium having such software or computer program recorded on it is a computer program product. The use of the computer program product in the computer system 1300 preferably effects an apparatus for in vivo bioprinting.

In some instances, the application programs 1333 may be supplied to the user encoded on one or more CD-ROMs 1325 and read via the corresponding drive 1312, or alternatively may be read by the user from the networks 1320 or 1322. Still further, the software can also be loaded into the computer system 1300 from other computer readable media. Computer readable storage media refers to any non-transitory tangible storage medium that provides recorded instructions and/or data to the computer system 1300 for execution and/or processing. Examples of such storage media include floppy disks, magnetic tape, CD-ROM, DVD, Blu-ray™ Disc, a hard disk drive, a ROM or integrated circuit, USB memory, a magneto-optical disk, or a computer readable card such as a PCMCIA card and the like, whether or not such devices are internal or external of the computer module 1301. Examples of transitory or non-tangible computer readable transmission media that may also participate in the provision of software, application programs, instructions and/or data to the computer module 1301 include radio or infra-red transmission channels as well as a network connection to another computer or networked device, and the Internet or Intranets including e-mail transmissions and information recorded on Websites and the like.

The second part of the application programs 1333 and the corresponding code modules mentioned above may be executed to implement one or more graphical user interfaces (GUIs) to be rendered or otherwise represented upon the display 1314. Through manipulation of the haptic interface 1302 and the mouse 1303, a user of the computer system 1300 and the application may manipulate the interface in a functionally adaptable manner to provide controlling commands and/or input to the applications associated with the GUI(s). Other forms of functionally adaptable user interfaces may also be implemented, such as an audio interface utilizing speech prompts output via the loudspeakers 1317 and user voice commands input via the microphone 1380.

FIG. 11B is a detailed schematic block diagram of the processor 1305 and a “memory” 1334. The memory 1334 represents a logical aggregation of all the memory modules (including the HDD 1309 and semiconductor memory 1306) that can be accessed by the computer module 1301 in FIG. 11A.

When the computer module 1301 is initially powered up, a power-on self-test (POST) program 1350 executes. The POST program 1350 is typically stored in a ROM 1349 of the semiconductor memory 1306 of FIG. 11A. A hardware device such as the ROM 1349 storing software is sometimes referred to as firmware. The POST program 1350 examines hardware within the computer module 1301 to ensure proper functioning and typically checks the processor 1305, the memory 1334 (1309, 1306), and a basic input-output systems software (BIOS) module 1351, also typically stored in the ROM 1349, for correct operation. Once the POST program 1350 has run successfully, the BIOS 1351 activates the hard disk drive 1310 of FIG. 11A. Activation of the hard disk drive 1310 causes a bootstrap loader program 1352 that is resident on the hard disk drive 1310 to execute via the processor 1305. This loads an operating system 1353 into the RAM memory 1306, upon which the operating system 1353 commences operation. The operating system 1353 is a system level application, executable by the processor 1305, to fulfil various high level functions, including processor management, memory management, device management, storage management, software application interface, and generic user interface.

The operating system 1353 manages the memory 1334 (1309, 1306) to ensure that each process or application running on the computer module 1301 has sufficient memory in which to execute without colliding with memory allocated to another process. Furthermore, the different types of memory available in the system 1300 of FIG. 11A must be used properly so that each process can run effectively. Accordingly, the aggregated memory 1334 is not intended to illustrate how particular segments of memory are allocated (unless otherwise stated), but rather to provide a general view of the memory accessible by the computer system 1300 and how such is used.

As shown in FIG. 11B, the processor 1305 includes a number of functional modules including a control unit 1339, an arithmetic logic unit (ALU) 1340, and a local or internal memory 1348, sometimes called a cache memory. The cache memory 1348 typically includes a number of storage registers 1344-1346 in a register section. One or more internal busses 1341 functionally interconnect these functional modules. The processor 1305 typically also has one or more interfaces 1342 for communicating with external devices via the system bus 1304, using a connection 1318. The memory 1334 is coupled to the bus 1304 using a connection 1319.

The application program 1333 includes a sequence of instructions 1331 that may include conditional branch and loop instructions. The program 1333 may also include data 1332 which is used in execution of the program 1333. The instructions 1331 and the data 1332 are stored in memory locations 1328, 1329, 1330 and 1335, 1336, 1337, respectively. Depending upon the relative size of the instructions 1331 and the memory locations 1328-1330, a particular instruction may be stored in a single memory location as depicted by the instruction shown in the memory location 1330. Alternately, an instruction may be segmented into a number of parts each of which is stored in a separate memory location, as depicted by the instruction segments shown in the memory locations 1328 and 1329.

In general, the processor 1305 is given a set of instructions which are executed therein. The processor 1305 waits for a subsequent input, to which the processor 1305 reacts to by executing another set of instructions. Each input may be provided from one or more of a number of sources, including data generated by one or more of the input devices 1302, 1303, data received from an external source across one of the networks 1320, 1302, data retrieved from one of the storage devices 1306, 1309 or data retrieved from a storage medium 1325 inserted into the corresponding reader 1312, all depicted in FIG. 11A. The execution of a set of the instructions may in some cases result in output of data. Execution may also involve storing data or variables to the memory 1334.

The disclosed in vivo bioprinting arrangements use input variables 1354, which are stored in the memory 1334 in corresponding memory locations 1355, 1356, 1357. The in vivo bioprinting arrangements produce output variables 1361, which are stored in the memory 1334 in corresponding memory locations 1362, 1363, 1364. Intermediate variables 1358 may be stored in memory locations 1359, 1360, 1366 and 1367.

Referring to the processor 1305 of FIG. 11B, the registers 1344, 1345, 1346, the arithmetic logic unit (ALU) 1340, and the control unit 1339 work together to perform sequences of micro-operations needed to perform “fetch, decode, and execute” cycles for every instruction in the instruction set making up the program 1333. Each fetch, decode, and execute cycle comprises:
a fetch operation, which fetches or reads an instruction 1331 from a memory location 1328, 1329, 1330;
a decode operation in which the control unit 1339 determines which instruction has been fetched; and
an execute operation in which the control unit 1339 and/or the ALU 1340 execute the instruction.

Thereafter, a further fetch, decode, and execute cycle for the next instruction may be executed. Similarly, a store cycle may be performed by which the control unit 1339 stores or writes a value to a memory location 1332.

Each step or sub-process in the processes of FIGS. 5 and 7 is associated with one or more segments of the program 1333 and is performed by the register section 1344, 1345, 1347, the ALU 1340, and the control unit 1339 in the processor 1305 working together to perform the fetch, decode, and execute cycles for every instruction in the instruction set for the noted segments of the program 1333.

The method of controlling arm 200 and bioprinter 300 may alternatively be implemented in dedicated hardware such as one or more integrated circuits performing the functions or sub functions of the described methods. Such dedicated hardware may include graphic processors, digital signal processors, or one or more microprocessors and associated memories.

### INDUSTRIAL APPLICABILITY

The arrangements described are applicable to the medical industries and particularly for in vivo bioprinting.

The foregoing describes only some embodiments of the present invention, and modifications and/or changes can be made thereto without departing from the scope and spirit of the invention, the embodiments being illustrative and not restrictive.

In the context of this specification, the word “comprising” means “including principally but not necessarily solely” or “having” or “including”, and not “consisting only of”. Variations of the word “comprising”, such as “comprise” and “comprises” have correspondingly varied meanings.

1. An apparatus comprising:
a soft robotic arm having actuators for controlling movements of a distal end of the soft robotic arm; and
a three-dimensional (3D) bioprinter attached to the distal end of the soft robotic arm, the 3D bioprinter having a print nozzle and soft microtubule artificial muscles (SMAMs) for controlling movements of the print nozzle.

2. The apparatus of claim 1, comprising:
a hydraulic system for controlling the actuators and the SMAMs by injecting or extracting fluid to or from fluid transmission tubes, wherein the fluid transmission tubes are in communication with the actuators and the SMAMs.

3. The apparatus of claim 2, comprising:
a computer system for controlling the hydraulic system.

4. The apparatus of any one of claims 1 to 3, wherein each of the actuators includes an elastic tube and means for restricting a radial expansion of the elastic tube, the means having a conduit within which the elastic tube is disposed.

5. The apparatus of claim 4, wherein each of the actuators and means corresponding lengthen and contract together.

6. The apparatus of any one of claims 1 to 5, wherein the 3D bioprinter includes a printing guide having a printing area, wherein the print nozzle moves over the printing area when actuated by the SMAMs.

7. The apparatus of any one of claims 1 to 6, wherein one of the SMAMs actuates the print nozzle to move longitudinally.

8. The apparatus of any one of claims 1 to 7, wherein the 3D bioprinter includes a protective sheath configured for protecting a distal end of the 3D bioprinter.

9. A method of controlling the soft robotic arm of any one of claims 1 to 8, the method comprising:
receiving a user input to move the distal end of the soft robotic arm to a direction;
determining lengths of the respective actuators to move to the direction; and
transmitting control signals to actuate the actuators based on the determined lengths of the actuators.

10. The method of claim 9, wherein the lengths determination uses a kinematic model representing the relationship between a position and an orientation of the distal end of the soft robotic arm and the lengths of the actuators.

11. A method of controlling the 3D bioprinter of any one of claims 1 to 8, the method comprising:
receiving a predetermined path;
determining a sequence of movements of the print nozzle based on the predetermined path;
determining a sequence of lengths of respective SMAMs based on the determined movement sequence; and
transmitting control signals to actuate the actuators based on the determined sequence of lengths.

12. The method of claim 11, wherein the lengths sequence determination uses an inverse kinematic model representing a relationship between a position of the print nozzle and the length of each SMAM.

13. The method of claim 11 or 12, wherein the actuation of the hydraulic system uses a hysteresis model of the SMAMs.

14. The method of claim 11 or 12, wherein the actuation of the hydraulic system uses a neural network.

15. The method of claim 14, wherein the neural network receives the position of the print nozzle and pressures of the SMAMs to determine the control signals for actuating the hydraulic system.

16. A computer program product comprising a computer program application that is executable by a processor, wherein the processor, when executing the computer program application, performs the method of any one of claims 9 and 10.

17. A computer program product comprising a computer program application that is executable by a processor, wherein the processor, when executing the computer program application, performs the method of any one of claims 11 to 15.

18. The method of claim 9 or 11 of controlling the soft robotic arm of claim 2, wherein the control signals are transmitted to actuate the hydraulic system.
